(19) United States
(12) Patent Application Publication
(10) Pub. No.: US 2021/0154156 A1
(43) Pub. Date: May 27, 2021

(54) APPLICATION OF COMPOUND IN INHIBITING AB ACCUMULATION AND TREATING ALZHEIMER'S DISEASE

(71) Applicant: NORTHEAST NORMAL UNIVERSITY, Changchun City, Jilin (CN)

(72) Inventors: Xiaomeng LI, Changchun City (CN); Lichun WANG, Changchun City (CN); Xiaowan WANG, Hangzhou (CN); Jiale LI, Tacheng City (CN); Chuanjian LAN, Sanhe City (CN); Jiang LI, Changchun City (CN)

(73) Assignee: NorthEast Normal University, Changchun City, Jilin (CN)

(21) Appl. No.: 17/044,109

(22) PCT Filed: Jan. 14, 2019
(86) PCT No.: PCT/CN2019/071554

§ 371 Date: Sep. 30, 2020

(30) Foreign Application Priority Data
Apr. 24, 2018 (CN) 201810371294.7

(51) Int. Cl. A61K 31/122; A61P 25/28

(52) U.S. Cl. CPC A61K 31/122; A61P 25/28

(57) ABSTRACT

Disclosed is application of emodin in inhibiting amyloid β-protein (Aβ) accumulation, improving cognitive and memory abilities, and reducing senile plaques. Alzheimer's disease (AD) is a neurodegenerative disease which is characterized in that nerve cell fibers are entangled. Aβ plaques are deposited, and a cognitive function is impaired to a pathological feature, and extracellular toxicity accumulation of Aβ is considered as one of main links of AD pathogenesis. In the present invention, it is discovered that emodin can obviously inhibit accumulation of an Aβ42 monomer and formation of fibers and inhibit formation of a β secondary structure, can improve spatial memory learning ability of an AD mouse, and can obviously clear amyloid plaques in APP/PS1 double-transpenic AD model mouse brain tissues. It shows that emodin can be applied to treatment of AD, and research and development of related medicaments.

### TECHNICAL FIELD

The invention belongs to the technical field of medicine and specifically relates to the application of emodin to antagonize the accumulation of beta amyloid 1-42 (Aβ42) as a target to prepare drugs for treating Alzheimer's disease.

### BACKGROUND OF THE TECHNIQUE

Alzheimer's Disease (AD) is clinically manifested as psychomotor abnormalities, language disorders, and cognitive and memory impairment, is a common progressive neurological disorder that occurs in the elderly According to the “2009 World Alzheimer's Report,” the number of people suffering from the disease will surge to 1.5 billion by 2050^{[1]}.

The most widely accepted pathogenesis of AD is the Aβ doctrine marked by the formation of β-amyloid oligomers and fibrous precipitates^{[2]}. Under normal physiology, the production and degradation of Aβ, which is hydrolyzed by β amyloid precursor protein (APP) by β and γ cleavage enzymes, is in equilibrium. Under pathological conditions, APP metabolism is abnormal, and the production of Aβ increases sharply. Because it cannot be cleared in time, it accumulates in the brain. The formation of toxic oligomers makes nerve cell fibrillary tangles, astrocytes and microglia proliferate. The release of inflammatory factors (such as TNF-α, IL-6, IL-2) causes a cascade reaction, which in turn will enhance the production of Aβ^{[3]}, triggering neurodegenerative diseases. Therefore, clearing the accumulation of Aβ in the brain has become an important way to treat the disease^{[4]}.

At present, there are no specific drugs for the treatment of AD. Existing drugs for the treatment of AD, such as cholinesterase inhibitor donepezil hydrochloride, non-steroidal anti-inflammatory drug aspirin, and calcium antagonist nimodipine can only slow down the progression of AD and improve symptoms, but cannot prevent the disease development. Based on the Aβ hypothesis, design β-secretase and γ-secretase inhibitors to reduce the production of Aβ, but the reduction of secretase itself is accompanied by greater side effects; immunotherapy, Aβ antibodies, peptides, etc. design themselves for how antibodies and peptides pass through the blood in the brain. The effective accumulation of barriers in the brain and side effects is another challenge for the treatment of Alzheimer's disease.

Epidemiological studies have found that some natural active ingredients in fruits, vegetables and Chinese herbal medicines can reduce the risk of AD^{[5]}. Curcumin can inhibit the accumulation of Aβ protein in vitro and eliminate the senile plaques in the brain of AD mice, reducing the toxicity of Aβ oligomers^{[6]}. It was also found that resveratrol, the main polyphenol component of grapeseed extract, can also inhibit the accumulation of Tg2576 Aβ and improve memory function impairment^{[7]}. This evidence indicates that the development of small molecule compounds that inhibit the accumulation of Aβ and reduce its toxicity can become a promising research direction for AD treatment.

Large flavin (English name Emodin, chemical name 1,3,8-trihydroxy-6-methylanthracene-9, 10-dione, molecular formula C15H10O5) is derived from the dry rhizome and root of Polygonum cuspidatum. The rhizome of rhubarb, a plant-type medicine. As of March 2018, there were about 165 foreign literature searches on research reports of emodin monomer in Alzheimer's disease. Most studies focused on the exploration of the role of emodin on Alzheimer's-related signaling pathways. There is no report on the application of antagonizing Aβ protein accumulation as a target to treat AD. The present invention is based on AD amyloid hypothesis to systematically study the effect of emodin in AD treatment through in vivo and in vitro experiments.

### REFERENCES

1. World Alzheimer's Report. 2009, Martin Prince. Jim Jackson et al. 2009

2. Hardy J, Selkoe D J. The Amyloid Hypothesis of Alzheimer's Disease: Progress and Problems on the Road to Therapeutics. Science et al. 2002: 297: 353-356

3. Mehhlhom G, Holborn M, et al., Induction of Cytokines in Glial Cells Surrounding Cortical beta-amyloid Plaques in Transgenic Tg2576 mice with Alzheimer Pathology, Int. J Dev. Neurosci. 2000. 18 (4-5): 423-31

4. Wang J, Ho L, et al. (2006a) Moderate consumption of Cabernet Sauvignon attenuate A beta neuropathology in a mouse model of Alzheimer's disease. FASE BJ20: 2313-2320

5. Assuncao M, Santos Marques M J, et al. Green tea averts age-dependent decline of hippocampal signaling systems related to antioxidant defenses and survival. Free Radic Biol Med. 2010. 48: 831-838

6. Venus Singh Mithu, Bidyut Sarkar, et al. Curcumin alters the salt bridge—containing turn region in amyloid betal—42 aggregates. JBC. 2014. 289(16): 11122-11131

7. Liu P, L J, et al. Grape seed polyphenolic extract specifically decreases Aβ56 in the brains of Tg2576 mice. J Alzheimer Dis. 2011. 26(4): 657-66

### SUMMARY OF THE INVENTION

1. The application of emodin in the treatment of Alzheimer's disease by antagonizing the accumulation of Aβ protein 1-42.

2. Application of emodin in the preparation of drugs for the treatment of Aβ amyloidosis in the brain.

3. Compared with the prior art, the beneficial effect of the present invention is that: the present invention is based on the hypothesis of AD amyloid, and conducts experiments in vitro and in vivo to systematically evaluate the effect of emodin in AD treatment research. It is confirmed that emodin can affect the secondary structure of Aβ protein, monomer polymerization, neurotoxicity of oligomers, formation of fiber structure, and Aβ amyloid in B6C3-Tg of Alzheimer's APP/PS1 double transgenic AD mice. The formation of protein deposition has a significant inhibitory effect, and can significantly improve the learning and memory ability of AD mice.

4. The present invention uses APP/PS1 double-transgenic mice as an animal model for the curative effect of emodin on Alzheimer's disease, and feeds them for a long time by gavage, and determines the effect of emodin on the clearance of Aβ protein senile plaques in the brains of AD mice and its effects on improved learning and memory function.

### ATTACHED FIGURES

FIG. 1 The results of the Thioflavin T test (ThT) that emodin inhibits the aggregation of Aβ42 monomers and the MTT test that inhibits the neurotoxic injury mediated by Aβ42 oligomers

FIG. 2 Transmission electron microscopy (TEM) of emodin inhibiting Aβ42 monomer to form fiber structure.

FIG. 3 Circular dichroism spectroscopy detects the results of emodin inhibiting the formation of Aβ42 monomer β sheets (CD)

FIG. 4 Depolymerization of the formed Aβ fiber structure by emodin (left ThT, right TEM)

FIG. 5 The water maze test detects the improvement of memory ability of APP/PS1 transgenic mice by emodin (Morris Water Maze).

FIG. 6 Water maze test to detect the improvement of memory ability of APP/PS1 transgenic mice by emodin (Y Maze)

FIG. 7 Thioflavin S staining to detect the effect of emodin on clearing senile plaques (ThS) in the brain of APP/PS1 transgenic mice

FIG. 8 Congo red staining to detect the effect of emodin on clearing senile plaques in the brain of APP/PS1 transgenic mice (Congo Red)

### EXPERIMENTAL PROGRAMS

Hereinafter, in conjunction with the accompanying drawings, the embodiments of the present invention are used to further illustrate the essence of the present invention, but the present invention is not limited thereto. For those of ordinary skill in the technical field to which the present invention belongs, without departing from the concept of the present invention, several simple inference and substitutions can be made, which should be regarded as belonging to the protection scope of the present invention.

### Example 1

### The Thioflavin T Test (ThT) that Emodin Inhibits Aβ42 Monomer Aggregation and the MTT Test that Inhibits Aβ42 Oligomer-Mediated Neurotoxicity Damage

The protein A beta protein was provided by Nanjing Elutrian Biological Co. Ltd. and the purity was greater than 95%. The compound macroflavin was provided by Shanghai Yuan-Ye biological company. The Aβ monomer powder was stored at −80° C., standing for 30 minutes at room temperature for the first time, then diluted with hexafluoroisopropanol (HFIP) to 1.0 mg/mL. It stood for at least 2 hours, carrying out ultrasonic 30 minutes to destroy the existing multi-polymer structure, standing and drying at low temperature, was immediately stored at −20° C. Thioflavin T was purchased from Sigma (cargo number T3516-5 G). 0.032 g of THT powder was weighed and dissolved in 20 mL of 20 mM Tris-HCl (pH 7.4) solution to obtain a 5 mM THT mother liquor, and was diluted to 5 uM when used.

When in use, the HFIP treated Aβ protein and the compound macroflavin were respectively dissolved by 100 ul, PBS buffer (100 nm phosphate, 10 mM NaCl, pH 7.4) was diluted, the final concentration of Aβ in the mixed solution was 20 uM, the concentration of the monomer drug was 0 uM control group and 10 uM (Low), 20 uM (Middle), and the 40 uM (High) experiment group, and each sample was 30 ul in total. The preparation method was comprised of the following steps: slowly shaking and incubating the prepared mixed solution at 37° C. for 24 hours, adding 270 ul 5 uM ThT, detecting by a fluorescence analysis tester, exciting a wavelength Ex=430 nm, emitting wavelength Em=480 nm to measure fluorescence values, testing each sample for 5 times, and taking an average value.

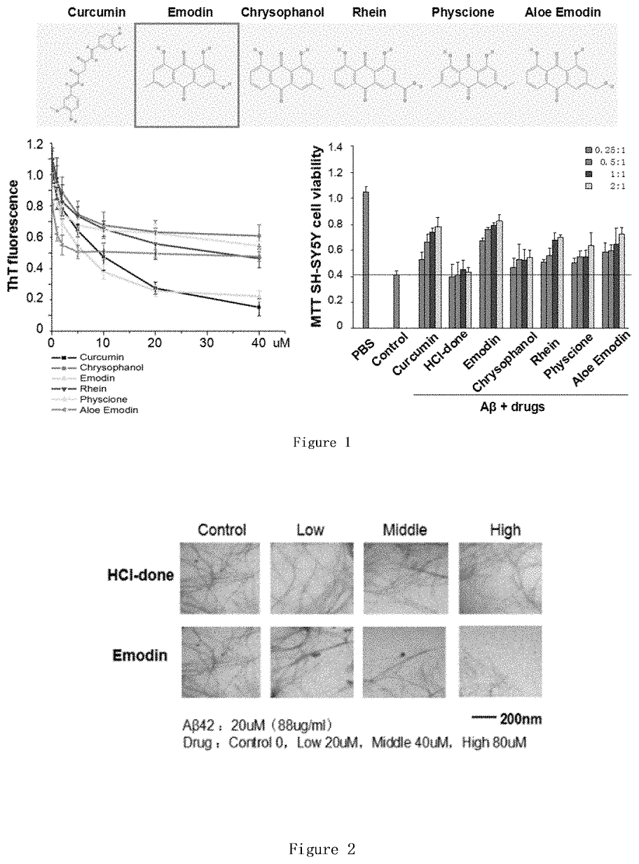

Using the above method, the results of the fluorescence intensity of each group detected are shown in FIG. 1, bottom left. It can be seen that compared with the control group without medicine, the fluorescence intensity of the experimental group with emodin compound was significantly decreased and was concentration-dependent, which was similar to the result of curcumin, a positive drug that inhibits Aβ protein aggregation. Several other emodin structurally similar compounds were not as effective as emodin. Test results show that the compound emodin can significantly inhibit the effective aggregation of Aβ protein monomers, and can be used to prevent and treat Alzheimer's disease.

The cells used in the Aβ42 oligomer-mediated neurotoxicity damage experiment were human neuroma blasts SH-SY5Y, incubated in DME medium of 10% FBS at 37° C. The control group without Aβ protein and the experimental group with added drug were cultured overnight at 37° C. with slow shaking, and transferred to a 96-well cell culture plate containing SH-SY5Y. The final concentration of Aβ protein was 1 uM and the final concentration of drug was 0.25 uM. 0.5 uM, 1 uM and 2 uM, three parallel holes for each concentration, action time 3 hours, MTT method 490 nm wavelength tested 5 times to analyze cell proliferation.

Using the above method, the results of the detected absorbance values of each group are shown in FIG. 2, lower right. It can be seen that compared with the control group without drug, the absorbance value of the experimental group with emodin compound increased significantly, and the drug emodin significantly inhibited the aggregation of Aβ protein to form oligomer-mediated neurocytotoxicity reactions. The effect was stronger than that of the positive control curcumin when the concentration was low. While another group of cholinesterase inhibitor drugs Arishen (donepezil hydrochloride, HCl-done) had no effect on the cytotoxicity caused by the aggregation of Aβ protein, the effects of other drugs having similar structures to emodin were not as good as emodin. The test results showed that the compound emodin can significantly inhibit neurocytotoxicity mediated by Aβ protein monomer aggregation, and can be used to prevent and treat Alzheimer's disease.

### Example 2

### Transmission Electron Microscopy (TEM)

The preparation was the same as that of Example 1. Then, the Aβ protein sample was incubated at 37° C. for 24 hours, dropped on the surface of a 300 mesh carbon film copper mesh, dried in air, dyed with 2% (w/v) phosphotungstate sodium salt negative for 30 seconds, standing at room temperature for about 4 hours, then the JEM-100CXII transmission electron microscope system (JEOL Inc., Tokyo, Japan) was used to detect and take pictures with an acceleration voltage of 80 kV.

The observation results are shown in FIG. 2. Compared with the unmedicated ingredients, the experimental group (EMO) with emodin compound added to the experimental group (EMO) Aβ protein formed fiber density, the length and width decreased significantly and was concentration-dependent. The other group of cholinesterase inhibitor drugs Arishen (donepezil hydrochloride) had no effect on the inhibition of the fiber formation of the Aβ protein. The test results showed that the compound emodin can significantly inhibit Aβ protein monomer forms a fibrous structure, which can be used to prevent and treat Alzheimer's disease.

### Example 3

### Circular Dichroism Spectrum Experiment (CD) of Emodin and Aβ Protein

The A beta monomer was mixed with a drug, the final concentration of Aβ was 20 uM, the final concentrations of the drug were 0 uM, 10 uM, 20 uM and 40 uM respectively, and after 12 hours incubation at 37° C., a J-810 spectrometer (Jayasco, Japan) was used for detection. The wavelength range was 190-250 nm, the resolution was 0.5 nm, the spectrum width was 2 nm, the scanning speed was 100 nm/min, the response time was 1 s, and the measuring temperature was normal temperature. The solution was added to a quartz cuvette with an optical path of 0.1 mm for measurement, and each experiment was repeated three times. Subtracting the buffer curve from each scan curve as the experimental result, each experiment was repeated three times, and the average value was given.

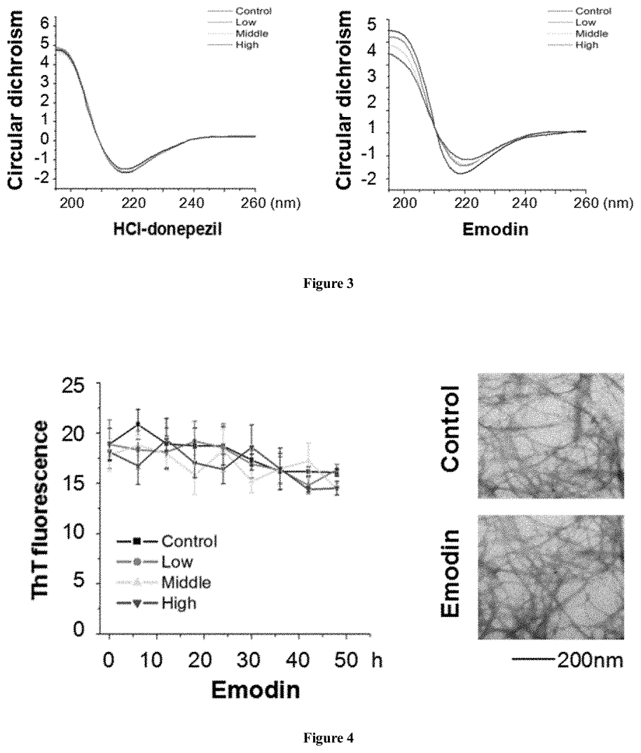

Using the software of Jascow32 provided by the instrument manufacturer to analyze the data (FIG. 3), the Aβ protein monomer mainly formed the β-sheet structure. Compared with the control group without flavin, the addition of emodin could significantly reduce the formation of the β-sheet structure. The concentration was dependent, and another group of cholinesterase inhibitor drugs Arishen (donepezil hydrochloride) had no effect on the formation of the monomer β-sheet structure of Aβ protein. It showed that the compound emodin can obviously inhibit the formation of the secondary β-sheet structure of Aβ protein monomer, and can be used to prevent and treat Alzheimer's disease.

### Example 4

### Depolymerization of Large Flavin on the Formed Aβ Fibrous Structure

The preparation was the same as that shown in Examples 1 and 2, except that the Aβ42 protein was first incubated at 37° C. for 48 hours, then the drug effect was added, the sample was taken at −20 ° C. every 6 hours, after 48 hours, using all of the samples for ThaT and TEAM tests.

With the above method, the fluorescence intensity results of each group were shown in FIG. 4, left; TEM results were shown in FIG. 4, right. It can be seen that compared with the non-dosing CONTROL group, the fluorescence intensity of the experimental group added with emodin compound remained basically unchanged, and the TEM results after 48 hours of treatment showed that emodin could not depolymerize the formed Aβ fibers.

### Example 5

### Behavioral Experiment of Large Flavin on App/PS1 Transgenic Mice

The wild-type (WT) and APP/PS1 double transgenic mice used in the experiment of the present invention were purchased from the Model Animal Research Institute of Nanjing University, and the mouse genes were identified in strict accordance with the PCR method provided. The 8-month-old APP/PS1 transgenic mice were set as the emodin administration group (low-concentration group EMO L, high-concentration group EMO H), Arishen administration group, and physiological saline as the Control group 1, and wild type (WT) mice was Control group 2, the nourishing blood serum brain drug treatment group (YX) found in our laboratory was the positive Control group 3, and continued intragastric administration for 2 months.

In the Y maze experiment, three black striped long arms were marked A, B, and C with the same appearance were combined in a Y shape. The mouse was put in the A arm, and recorded as an error if it turned back from the other long arm during free movement, such as ABA, BCB, etc., recalculated with this arm as the starting point, such as ABACBABCBA. The experiment time for each mouse lasted 8 minutes. When the experiment was completed, the Y labyrinth was cleaned and disinfected with alcohol, and the experiment was performed on the next mouse after drying.

In the Morris water maze experiment, the mouse water maze training was two days (data not recorded). The mice were placed into the pool from 4 entry points (quadrants) facing the pool wall. The platform was hidden 1 cm underwater, and the water temperature was kept at 23° C. , and the training lasted for 2 minutes. If the mouse failed to find the platform within 2 minutes it would be guided to the hidden platform with tools and stayed for 20 seconds, and then proceed to the next training. The experiment phase started from the third day, and the experiment was continued for 6 days. Each mouse was tested once a day. During the experiment, the mice were respectively placed into the pool from 4 entry points (quadrants) facing the pool wall.

The time was recorded from the mice entering the water to the mice finding and staying in the underwater hidden platform. That was called as the incubation period. On the eighth day, the platform was removed, and each mouse was launched from the farthest position of the original platform and swam freely for 2 minutes. The swimming time of the animal in the target quadrant (the area where the platform located), the number of times of traversing the platform, the path and other main parameters were tracked and recorded by the miniature camera and analyzed.

The results of the Morris water maze experiment showed that after emodin treatment the incubation period for AD mice to find a platform was significantly reduced. Compared with wild mice (WT) and AD intragastric saline group (Control), the training experiment started on the third day. The incubation period of AD mice treated with emodin gradually decreased (FIG. 5, Bottom left). After the platform was removed, the mice were subjected to a positioning navigation experiment. The results showed that the number of times AD mice in the emodin treatment group crossed the virtual platform was significantly improved (FIG. 5, Lower right). The effect was similar to that of the positive control drug for nourishing serum and brain, while in comparison, the Arishen drug group had a limited effect on improving symptoms of AD mice. Based on the above behavioral results, it can be seen that emodin can significantly improve the learning and memory ability of AD mice. The results also indicated that emodin can be used in the development of drugs for the prevention and treatment of Alzheimer's disease.

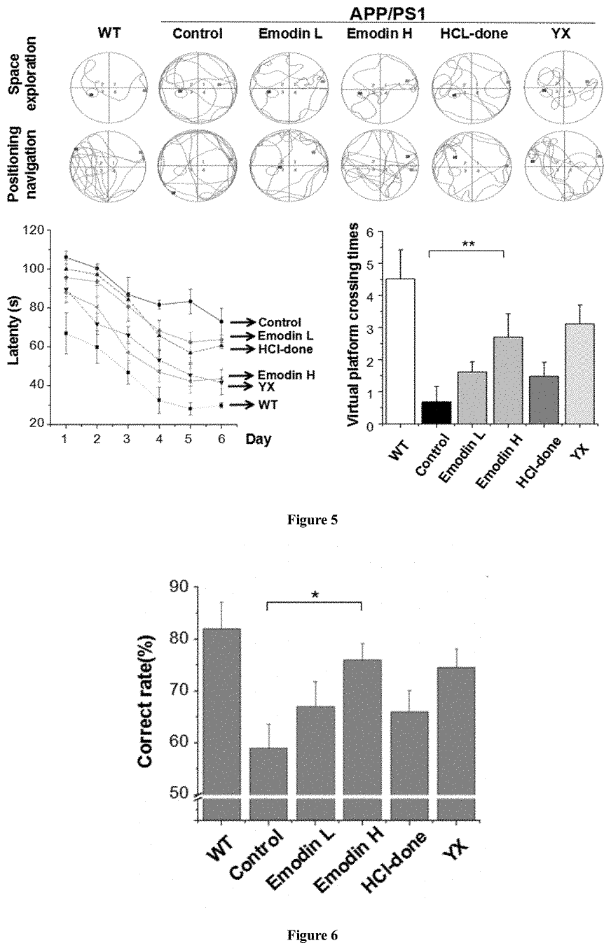

The results of the Y maze experiment are shown in FIG. 6. The correct rate of wild mice (WT) is about 82%. However, the correct rate of Y maze experiment on AD rats given by intragastric saline was only about 58%. After two months of emodin treatment, the correct rate of Y fans in AD mice increased significantly. The effect was slightly due to the serum-raising brain drug treatment group, which was better than the Arishen treatment group, indicating that emodin can improve the cognitive and memory ability of AD mice. It has the potential to become a preventive and therapeutic drug for Alzheimer's disease.

### Example 6

### Pathological Experiment of the Effect of Emodin on the Brain Tissue of APP/PS1 Transgenic Mice

10-month-old AD mice were anesthetized with 0.4% pentobarbital sodium. Blood was taken by perfusing the atria. 4% paraformaldehyde was perfused for whole body fixation. The brain tissue was taken and weighed. The hemibrains were put into 4% paraformaldehyde solution and used for histopathology. The other half was stored in a cryotube at −80° C. for biochemical analysis. The brain tissues fixed with 4% paraformaldehyde were embedded in wax and sliced. The plaques in the cerebral cortex and hippocampus were stained with Thioflavin S and Congo Red. Pathological images were obtained by digital microscope and fluorescence microscope and quantitatively analyzed.

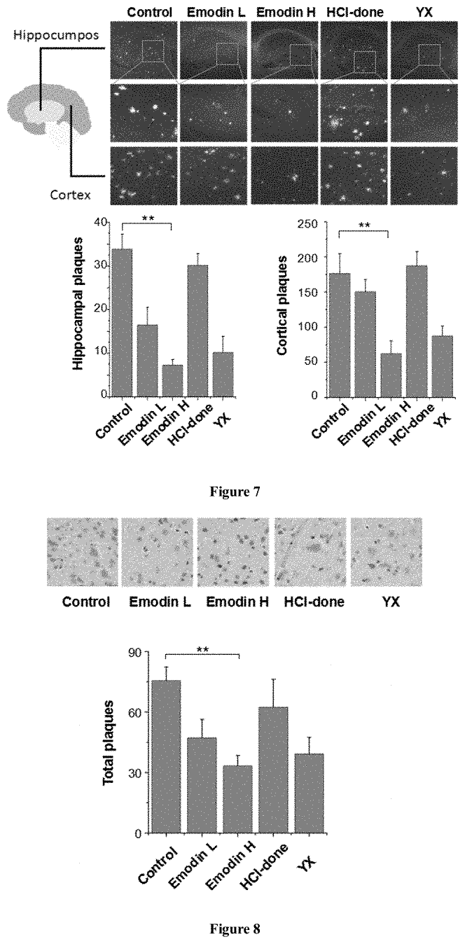

The results of Thioflavin S staining and Congo red staining of brain tissue are shown in FIGS. 7 and 8. The number of stained Aβ deposition plaques in the emodin treatment group in the cortex or hippocampus area was compared with that of the control with only normal saline. For AD mice, all showed a statistically significant reduction and the effect was slightly better than that of the positive control group nourishing blood serum brain drug treatment group, while the Arishen drug group had no significant effect on the brain tissue plaque deposition of AD mice. The changes indicated that emodin can inhibit the deposition of Aβ protein in brain tissue and can be used in the development of drugs for the prevention and treatment of Alzheimer's disease.

1. The application of the compound macroflavin in the aspect of antagonizing beta amyloid protein 1-42 for treating senile dementia is protected.

2. The senile dementia of claim 1, in particular Alzheimer's disease, is Alzheimer's disease.

3. The invention relates to application of protection compound macroflavin in research and development of Alzheimer's disease

4. The protection compound macroflavin is used for inhibiting Alzheimer's disease treatment and drug research and development in the aspect of inhibiting Aβ 1-42 monomer accumulation to form oligomer-mediated nerve cell toxicity damage

5. The protection compound macroflavin is formed by folding a Aβ 1-42 monomer beta, inhibiting the accumulation of Aβ1-42 monomer to form the Alzheimer's disease treatment and drug research and development of the oligomer and the fiber

6. The invention provides a therapeutic effect and related drug research and development in the aspect of inhibiting Aβ protein accumulation to form Alzheimer's disease of senile plaques.

7. The invention provides an Alzheimer's disease treatment effect for improving cognitive memory capacity disorder and related drug research and development.

8. The protection compound macroflavin and the analogues thereof have a molecular structure basis in Alzheimer's disease treatment and drug research and development applications, and the molecular structure of the large flavin is protected from the action of Alzheimer's disease and drug research and development. The chemical name of the emodin is Emodin, and the name is 518-82-1; Schuttggeb et al. Emodol et al. Frankula Emodin; Rheumin Emodin, International Compound Identification Inchi=1 S C15H10O 5/C1-6-2-8-12 (10 (17) 3-6) 15 (20) 13-9 (14 (8) 19) 4-7 (16) 5-11 (13) 18/H2-5, 16-18 H, 1H3At the same time, the molecular weight is 270.24 g/mol, and the application of the compound molecule with the large flavin similar structure in the treatment and drug research and development of Alzheimer's disease is protected. A similar compound formed by the simple substitution of the three hydroxyl groups of the large-flavin molecule or the change of the structure caused by the addition or addition of the group—CH2—, is considered to be the protection scope of the invention.
